(19) United States
(12) Patent Application Publication
(10) Pub. No.: US 2005/0080482 A1
(43) Pub. Date: Apr. 14, 2005

(54) Graft coupling apparatus and methods of using same

(76) Inventor: Craig Bonsignore, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/685,374

(22) Filed: Oct. 14, 2003

(51) Int. Cl. A61F 2/06

(52) U.S. Cl. 623/1.35

(57) ABSTRACT

This invention relates to devices and methods for sutureless-sealing of vessels. According to the invention there is provided a graft coupling device having cooperating interior and exterior anchors for sealing a first vessel and a second vessel.

### BACKGROUND OF THE INVENTION

I. Field of the Invention

The present invention relates to medical devices and methods for joining two vessels. More particularly, the present invention relates to devices and methods for using grafts, stents and other endovascular devices as a coupling device for joining two vessels.

II. Description of the Related Art

Endovascular grafts, stents and like devices are known in the art for aiding, repairing or bypassing blood flow through vascular vessels. As will be appreciated, these prior art devices require skill and precision during surgery to deliver and properly couple the device to the vasculature of the patient. Take bypass grafts for example. Aside from the difficulties associated with delivering the graft to the proper location, heretofore, in typical femoral-femoral or femoral-popliteal bypasses, for example, the bypass graft must be manually sutured to the native vessel. Surgically suturing the graft is a tedious and time consuming process, requiring substantial skill and experience to achieve a secure and leak-free coupling. Undesirable results, such as leaks, are not uncommon, and require the suturing to be modified or supplemented. The adverse consequences posed by suturing are clear drawbacks with these prior art grafts and like devices.

Accordingly, there exists a long-felt, yet unresolved need in the art for improved devices and methods for endovascular treatment and repairs, such as coupling grafts and bypass grafts and the like to native vessels, without the need for suturing.

### SUMMARY OF THE INVENTION

The present invention overcomes the practical problems described above and offers new advantages as well. One object of the present invention is to provide a sutureless method of joining two vessels. Another object of the present invention is to provide a sutureless method of coupling an artificial graft to a native vessel. Another object of the present invention is to provide devices and methods for coupling vasculature end-to-end. Yet another object of the present invention is to provide devices and methods for coupling vasculature end-to-side.

These and other objects and advantages of the present invention may be realized by an artificial graft coupling device including internal and/or external anchors to seal the endovascular vessel and graft.

One advantageous feature of the present invention is the provision of alternative methods and devices for end-to-end coupling of an artificial graft or the like to a main vessel. In a preferred exemplary embodiment, the end-to-end coupling is achieved without inserting-the bypass graft or donor vessel into the feeding vessel. Another advantageous feature of the present invention is the provision of methods and devices for coupling a donor vessel to a main vessel, with or without the use of an artificial graft or the like. Yet another advantageous feature of the present invention is the provision of methods and devices for end-to-side coupling of an artificial graft and/or donor vessel to a main vessel.

In accordance with an aspect of the present invention, a device for coupling vessels comprises a main trunk, first and second stent-anchors associated with the main trunk and a graft extension extending from said main trunk. A bypass vessel is provided with an internal anchor and an external anchor, the internal anchor and the external anchor cooperating to seal said graft extension and the bypass vessel.

In accordance with another aspect of the present invention, a device for coupling vessels comprises a main vessel having an end, a bypass vessel having an end and a coupling graft disposed around the main vessel end and the bypass end. A main vessel internal stent-anchor is provided to seal the main vessel with the coupling graft and a bypass vessel internal stent-anchor is provided to seal the bypass vessel with the coupling graft; whereby the main vessel and the bypass vessel are held in fluid communication via said coupling graft.

Given the following enabling description of the drawings, the devices and methods according to the present invention should become evident to a person of ordinary skill in the art.

### BRIEF DESCRIPTION OF THE DRAWINGS

The present invention is described with reference to the accompanying drawings. In the drawings, like reference numbers indicate identical or functionally similar elements.

FIG. 1 is a plan view of an embodiment of a graft coupling device for end-to-side sutureless coupling of vessels according to the invention.

FIG. 2 is a plan view of another embodiment of a graft coupling device for end-to-side sutureless coupling of vessels according to the invention.

FIG. 3 is a plan view of an embodiment of a coupling device for end-to-end sutureless coupling of vessels according to the invention.

### DETAILED DESCRIPTION OF THE PREFERRED EMBODIMENT

The present invention is based, in part, on the discovery that internal and external stent-like structures may be adapted to serve as anchors and seals to facilitate sutureless coupling of two or more vessels. While the present invention will be described in connection with end-to-end and end-to-side anastamosis methods and devices, one of ordinary skill in the art will readily appreciate that the present invention may be adapted for numerous uses in a variety of fields.

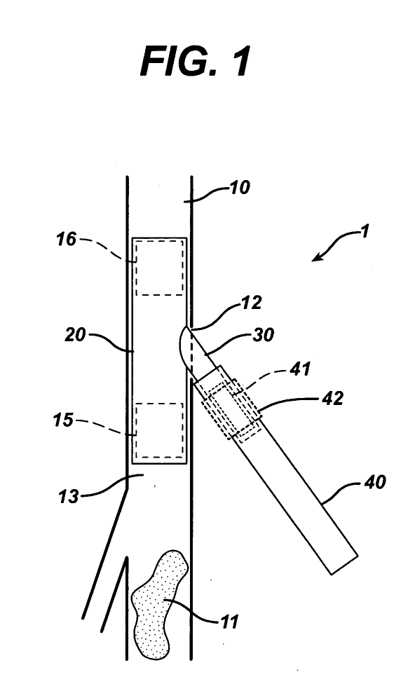

FIG. 1 depicts a presently preferred embodiment of a graft coupling device 1 according to the invention for end-to-side anastamosis. As depicted in FIG. 1, native vessel 10 has some disease or obstruction 11, requiring a bypass graft to preserve flow to some distal point in the vasculature. Such a situation is typical of femoral-femoral or femoral-politeal bypass procedures. As will be appreciated, in the case of end-to-side anastamosis, it is considered favorable to maintain flow distal to the site of graft attachment. In such a procedure, native vessel 10 will be accessed in an open procedure to allow a surgeon to make an incision 12 in side wall of vessel 10. With direct access to vessel 10 available, the surgeon places main trunk 20 of graft coupling device 1 in the interior 13 of vessel 10. In placing the device 1, the surgeon allows graft extension 30 to protrude from the incision 12.

Proximal and distal main trunk anchors 15, 16 serve to seal main trunk 20 to native vessel 10 proximally and distally to the incision 12 and graft extension 30. Anchors 15, 16 may be integral to main trunk 20, or alternatively, may be deployed in a secondary step. Preferably, anchors 15, 16 are interior anchors which expand radially or otherwise exert an outward force to frictionally seal main trunk 20 in place. Alternatively, exterior anchors which constrict radially or otherwise exert an inward force may be used.

Bypass graft 40 is extended over coupling graft extension 30. Preferably, graft extension 30 is sized such that its diameter approximates the inner diameter of bypass graft 40. An internal graft anchor 41 within bypass graft 40 exerts an outward force, while an external graft anchor 42 exerts and inward force. The opposing forces from these anchors secure the seal between the graft extension 30 and bypass graft 40. Alternatively, only an internal graft anchor 41 or external graft anchor 42 could be used. However, as will be appreciated by one of ordinary skill in the art, the opposing forces of using both anchors is preferably in sealing and ensuring the structural integrity of the device and its positioning is maintained.

The anchors according to the invention may be constructed of any suitable material and shaped in any suitable configuration. Preferably, the internal and external anchors exhibit stent-like characteristics. The internal and external stent-like anchors may both feature superelastic properties, or alternatively one or both may be plastically deformable.

In the case of a plastically deforming internal stent coupled with a superelastic external stent, the sealing pressure could be adjusted by balloon dilation of the internal stent. As the internal stent is ratcheted up in diameter, it increases the diameter of the external superelastic stent, thereby increasing the inward force it exerts on the graft-to-vessel seal.

In the case of a plastically deformable external stent, a similar effect may be achieved using a balloon internally to force expansion of the graft, vessel, and both anchor stents. Alternatively, the deformable external stent may be manually crimped or constrained in diameter to further reengage the superelastic internal stent, also thereby increasing sealing pressure.

In the cases wherein both the external and internal anchors are superelastic, their diameters would be set such that they would engage each other over a range of diameters with predictable or predetermined resultant sealing pressure. As such, the memory diameter of the external anchor would be smaller than the anticipated outer diameter of the external graft or vessel (depending on the procedure), while the memory diameter of the internal anchor would be larger than the anticipated inner diameter of the internal graft or vessel.

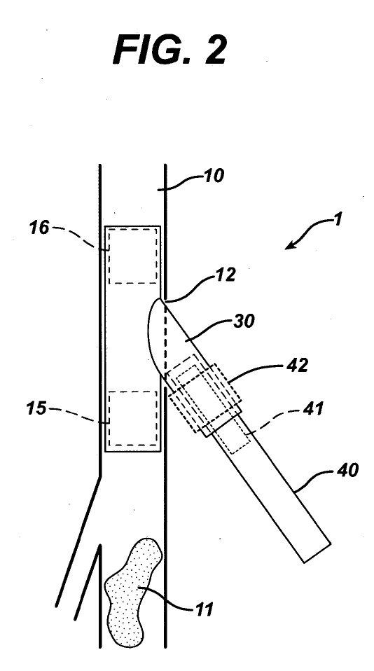

In an alternative exemplary embodiment for end-to-side anastamosis depicted in FIG. 2, the roles of the graft extension 30 and bypass graft 40 are reversed such that the graft extension is external and the bypass graft is internal. In accordance with this exemplary embodiment, the external stent-like anchor 42 could be integral with the graft extension 30 as an exoskeleton. This exemplary embodiment offers the possibility of a less invasive procedure in that the device and coupling can be achieved without external access to the vessel or junction site.

In operation, access to the femoral artery 10 (or other target vessel) is achieved through normal means well known in the art of endovascular procedures. A guide wire (not shown) is delivered to the site at which the bypass graft 40 is to join the main vessel 10. Once properly positioned, blood flow to the region is attenuated by some other endovascular means, for example, deployment of a balloon proximal to the site, preferably proximal to the wire access point. The desired junction site is deliberately punctured and dilated using endovascular techniques within the skill of the ordinary artisan. The graft coupling device 1, including main trunk 20, graft extension 30, and internal anchors 15, 16 and 41, is loaded in a constrained state into a delivery device (not shown). The delivery device is advanced along the guide wire to the desired site. Preferably, the delivery system allows positioning of a second guidewire which exits the graft extension 30.

The primary guidewire remains in the main vessel, while the secondary guidewire is navigated to exit the vessel 10 at the puncture site. Anchors 15, 16 are deployed to seal main trunk 20 of the coupling graft device 1 into the main vessel 10. The external anchor exoskeleton 42 in this exemplary embodiment is preferably plastically deformable. Accordingly, at this point in the procedure, a balloon is advanced along the secondary guidewire to the location of external anchor 42, and it is partially expanded to a diameter which allows it to accommodate bypass graft 40.

Bypass graft 40 is preferably navigated to the attachment site using some endoscopic other minimally invasive technique. Preferably, the device used to advance the graft must have the capability to locate and snare the second guidewire which was punctured through the vessel as described above. Once the guidewire is successfully located, the bypass graft 40 may be advanced into the graft extension 30 (which has preferably been partially expanded as previously described). Internal anchor 41 may now be advanced along the secondary guidewire and deployed inside bypass graft 40.

A final balloon inflation inside the internal anchor 41 fully expands external anchor 42 and fully engages the internal and external anchors to provide an adequate seal. Accordingly, at this point the vessels have been joined together without external manual access to the junction site. Blood flow may be restored when the opposite end of the bypass graft is properly terminated.

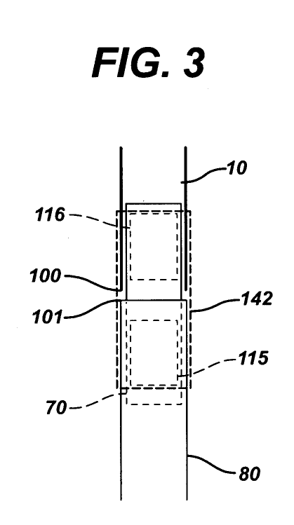

FIG. 3 depicts a presently preferred exemplary embodiment of the invention for end-to-end anastamosis. The device and procedure for end-to-end anastamosis according to this exemplary embodiment of the present invention is particularly preferred in cases where the bypass graft is a harvested vessel rather than an artificial graft. As depicted in FIG. 3, rather than insert one vessel inside another or suture their respective ends together, a straight tube-like coupling graft 70 is provided for joining the vessels ends. This coupling graft 70 is positioned inside of the two mating vessels.

As depicted, main vessel 10 is to be coupled to bypass graft or donor vessel 80. Accordingly, main vessel end 100 and donor vessel end 101 are positioned over coupling graft tube 70 and sealed in place with internal anchors 115, 116. Any suitable endovascular method for achieving this goal may be used. Other methods may be employed beside endovascular techniques. Once positioned, all or a portion of coupling graft tube 70 acts like an interior fluid coupling member, stent-anchor 142 which is positioned exterior of the mating vessels provides a force to provide fluid tight sealing from the exterior of the vessels. Preferably, tube 70 comprises a material suitable for use as an internal fluid carrying conduit and coupling device.

As will be appreciated, with this configuration of the present invention, neither main vessel 10 nor donor vessel 80 has to be fit within the other or have their respective ends 100, 101 sutured together.

Those skilled in the art will appreciate that various adaptations and modifications of the above-described preferred embodiments can be configured without departing from the scope and spirit of the invention. Therefore, it is to be understood that, within the scope of the appended claims, the invention may be practiced other than as specifically described herein.

1. A device for coupling vessels comprising:
a main trunk;
first and second stent-anchors associated with said main trunk;
a graft extension extending from said main trunk;
a bypass vessel;
an internal anchor and an external anchor; said internal anchor and said external anchor cooperating to seal said graft extension and said bypass vessel.

2. The device of claim 1 wherein said bypass vessel comprises an artificial graft or a donor vessel.

3. The device of claim 2 wherein said bypass vessel extends over said graft extension and wherein said internal anchor is disposed in said graft extension in an area at least partially overlapped by said bypass vessel.

4. The device of claim 3, wherein said external anchor is integral with said bypass vessel in an area which at least partially overlaps said bypass vessel.

5. The device of claim 2 wherein said graft extension extends over said bypass vessel and wherein said internal anchor is disposed in said bypass vessel in an area at least partially overlapped by said graft extension.

6. The device of claim 5 wherein said external anchor is integral with said graft extension in an area which at least partially overlaps said bypass vessel.

7. The device of claim 1 wherein said first and second stent-anchors are disposed in an interior area of said main trunk on opposite ends of said graft extension.

8. The device of claim 7 wherein said first and second stent-anchors exude an outward force to seal said main trunk in a main vessel.

9. The device of claim 8 wherein said first and/or second stent-anchors comprise a plastically deformable material.

10. A device for coupling vessels comprising:
a main vessel having an end;
a bypass vessel having an end;
a coupling graft disposed within said main vessel end and said bypass vessel end;
a main vessel internal stent-anchor configured to seal said main vessel with said coupling graft;
a bypass vessel internal stent-anchor configured to seal said bypass vessel with said coupling graft; whereby said main vessel and said bypass vessel are held in fluid communication via said coupling graft.

11. The device of claim 10, wherein said bypass vessel comprises a bypass graft or a donor vessel.

12. The device of claim 11, further comprising an external anchor for sealing said main vessel end and said bypass vessel end around said coupling graft.

13. The device of claim 12 wherein said main vessel internal stent-anchor and said bypass vessel internal stent-anchor exude an outward force to seal said coupling graft to said vessels.

14. The device of claim 13 wherein said stent-anchors comprise a plastically deformable material.

15. The device of claim 14 wherein said stent-anchors are ratchetable to increase a diameter of said stent-anchors.

16. The device of claim 15 wherein said stent-anchors are ratchetable by balloon expansion.

17. A method of performing end-to-side anastamosis comprising the steps of:
inserting a graft coupling device having a main trunk and a graft extension in a main vessel having an incision such that said graft extension protrudes from said extension;
sealing said main trunk to said main vessel with anchors;
placing a bypass graft over a portion of said graft extension; and
sealing said bypass graft and said graft extension with cooperating interior and exterior anchors.

18. A method of performing end-to-side anastamosis comprising the steps of:
delivering a first guide wire to a junction site in a main vessel;
puncturing said main vessel at said junction site;
delivering a graft coupling device having a main trunk and a graft extension having an external anchor to said junction site via said first guide wire;
positioning a second guide wire to exit said graft extension of said graft coupling device at said puncture site;
sealing said main trunk to said main vessel with interior anchors disposed on opposite ends of said puncture site;
advancing a balloon along said second guide wire to a location corresponding to said external anchor;
balloon expanding said external anchor to a diameter sufficient for accepting a bypass graft;
navigating a bypass graft to said junction site and into said graft extension;
advancing an internal anchor along said second guide wire; and
deploying said internal anchor, thereby sealing said extension graft and said bypass graft.

19. A method of performing end-to-end anastamosis comprising the steps of:
positioning a first vessel end and a second vessel end over a coupling graft; and
expanding an anchor in each of said ends for sealing said vessels with said coupling graft.
